(19) United States
(12) Patent Application Publication
(10) Pub. No.: US 2023/0384316 A1
(43) Pub. Date: Nov. 30, 2023

(54) MULTIPLEX METABOLIC MARKERS IN PLASMA FOR EARLY DETECTION OF AFRICAN AMERICAN PROSTATE CANCER

(71) Applicants: Baylor College of Medicine, Houston, TX (US); The United States of America as Represented by the Department of Veterans Affairs, Washington, DC (US); University of Florida Research Foundation, Incorporated, Gainesville, FL (US)

(72) Inventors: Arun Sreekumar, Sugar Land, TX (US); Bert W. O'Malley, Houston, TX (US); Jie Gohlke, Pearland, TX (US); Clifford Dacso, Houston, TX (US); Jeffrey Jones, Houston, TX (US); George Michailidis, Gainesville, FL (US); Nagireddy Putluri, Houston, TX (US)

(21) Appl. No.: 18/032,528

(22) PCT Filed: Oct. 20, 2021
(86) PCT No.: PCT/US2021/071935

§ 371 Date: Apr. 18, 2023

Related U.S. Application Data
(60) Provisional application No. 63/094,103, filed on Oct. 20, 2020.

(51) Int. Cl. G01N 33/68; G01N 33/574; G16H 50/20

(52) U.S. Cl. CPC G01N 33/6815; G01N 33/57434; G16H 50/20

(57) ABSTRACT

Embodiments of the disclosure include means of identifying and quantifying particular metabolite-based biomarkers for diagnosis and prognosis of prostate cancer in African American men, including at least Methionine, Homocysteine, Glutamic acid, Ornithine, and/or Inosine, and, in some cases, also N-Acetyl Aspartate, Glutamine, Sarcosine, Succinate, and/or Malate.

This application claims priority to U.S. Provisional Patent Application No. 63/094,103, filed Oct. 20, 2020, which is incorporated by reference herein in its entirety.

### TECHNICAL FIELD

Embodiments of the disclosure include at least the fields of molecular biology, cancer biology, metabolomics, and medicine, including oncology.

### BACKGROUND

Prostate cancer (PCa) is the most common solid tumor diagnosed among men of all racial/ethnic groups in the United States[1]. The global burden of this disease is rising. Its incidence and mortality rates are higher in African American (AA) men compared to white men and other ethnic groups. AA men have early onset of the disease with rapid progression to metastasis. Prostate specific antigen (PSA) levels in the serum are used clinically for the early detection of the disease [2]. Despite advances in screening for early detection of prostate cancer, a large percentage of men continue to be diagnosed with metastatic disease, including about 20% of men affected with a high mortality rate within the AA population. AA patients generally present higher PSA values across all stage, grade, and age categories [3]. Because of a lack of specificity of PSA for prostate cancer, needle biopsy is commonly used to verify the presence, extent, and characteristics of the tumor [4]. This has resulted in a rise in the number of “negative” biopsies being performed that, in addition to being invasive, are also associated with clinical complications [5]. At least half of the patients with needle biopsy tumor diagnosis need no immediate therapy. With the failure of PSA alone as reliable marker for prostate cancer detection, there is an urgent need to develop accurate biomarkers for early detection of this disease, especially in AA men.

Generally, local prostate cancer is divided into low-, intermediate-, and high-risk categories based on the clinical stage, PSA, and digital rectal examination results. Active surveillance (AS) is a recognized strategy recommended to low-risk prostate cancer with the intent of avoiding radical treatment. However, AA men had greater rates of Gleason score upgrading, lower rates of organ-confined cancers, and higher hazard rates of biochemical recurrence [6]. AA men are more likely to die from Low-Grade prostate cancer. Scientific findings support that prostate cancer grows more rapidly in AA men and earlier transformation from latent to aggressive prostate cancer occurs in AA men. By the time of diagnosis, the number of aggressive or rapidly growing tumors in AA is greater. Notably, there is a lack of predictive markers that can predict the likelihood of biochemical recurrence and/or castration resistant diseases of AA men with prostate cancer.

Metabolomic studies can identify disease-specific metabolic signatures and correlate them with clinically relevant outcomes [7, 8]. Unlike genomics, transcriptomics and proteomics, metabolomic analysis involves reduced complexity because of fewer endpoints (i.e., ˜3,000 compounds vs. 40,000 genes, 150,000 transcripts, and 1×10⁶ proteins) and allows the ability to assay composite downstream outputs from complex interconnected cellular processes [9]. Sub-classification and stratification of metabolomic changes may allow for the separation of pathologically similar cancers into biologically different groups based on prognosis [10]. In addition, metabolomics is unique in that it integrates genetic and non-genetic information. That non-genetic information includes lifestyle, diet, and the microbiome. Consequently, it may yield a more comprehensive picture of health disparity. A metabolite-based biomarker panel for prostate cancer early detection and/or predictive markers for prostate cancer progression is currently lacking to be integrated into the clinical setting.

The present disclosure provides a long-felt need in the art of providing early diagnosis or prognosis for AA men with prostate cancer or at risk for prostate cancer.

### BRIEF SUMMARY

The present disclosure is directed to systems, methods, and compositions for identifying and using biomarkers for predicting risk of prostate cancer and/or to assess a need for prostate biopsy. In some embodiments, the biomarkers include one or more metabolites obtained from a sample from an individual. Metabolites have been identified that can be used to diagnose prostate cancer, in specific cases. In some embodiments, these metabolites include Methionine, Homocysteine, Glutamic acid, Ornithine, and/or Inosine. In other embodiments, the metabolites include Methionine, Homocysteine, Glutamic acid, Ornithine, Inosine, N-Acetyl Aspartate, Glutamine, Sarcosine, Succinate and/or Malate. In specific embodiments, the metabolites include one or more of Melatonin, Aminobutyric Acid, Isoleucine, Adenosine, Methionine, Homocysteine, Valine, Kynurenine, Inosine, Arginine, Ornithine, Proline, Sarcosine, Putrescine, Glutathione, Glutamic acid, Glutamine, Kynurenic acid, Pyruvate, Lactate, Ketoglutarate, Acetyl Aspartic acid, Malate, 2-hydroxyglutarate, Succinate, and/or Fumarate. In specific embodiments, the metabolites include Carnitine, Cholic acid, Deoxycholic acid, Testosterone, 5-dihydrotestosterone, Estrone, Estradiol, Progesterone, and/or 25-hydroxyvitamin D3. In certain embodiments, the metabolites include one or more of the metabolites collectively in the aforementioned lists.

In particular embodiments, the metabolic biomarkers include metabolites that are differentially present in samples from individuals with prostate cancer as compared to samples from control individuals that do not have cancer or as compared to a standard. In other embodiments, the metabolite(s), in conjunction with one or more of prostate-specific antigen score, Gleason score, and/or body mass index, are used to predict incidence of prostate cancer or need for prostate biopsy. In specific embodiments, the individual in need of testing is an African American individual with West African ancestry. In some embodiments, the individual has over 70% West African ancestry based on a combination of genetic markers. In some embodiments, the ancestry of the individual is determined by SNP analysis.

In some embodiments, any method may comprise one or more of the steps of determining the grade and/or aggressiveness of the cancer, determining the stage and/or spread of the cancer, and/or identifying or predicting the cancer status of the individual. In some embodiments, the determinations and/or predictions are made using a machine learning classifier. In some embodiments, the machine learning classifier has been trained using biomarker levels obtained from individuals with prostate cancer and individuals that do not have prostate cancer. In specific embodiments, the machine learning classifier is a random forest.

In some embodiments, there is a method of treating an individual for prostate cancer comprising the step of administering an effective amount of treatment to an individual in need thereof when the individual has a change in the level of biomarkers comprising Methionine, Homocysteine, Glutamic acid, Ornithine, Inosine, or a combination thereof. In specific embodiments, the method of treating an individual for prostate cancer comprises the step of administering an effective amount of treatment to an individual in need thereof when the individual has a change in the level of biomarkers consisting of Methionine, Homocysteine, Glutamic acid, Inosine, or a combination thereof.

In a certain embodiment, there is a method of treating an individual for prostate cancer comprising the step of administering an effective amount of treatment to an individual in need thereof when the individual has a change in the level of biomarkers consisting essentially of Methionine, Homocysteine, Glutamic acid, Ornithine, Inosine, and/or a combination thereof.

In any method encompassed herein the individual may be classified as having at least 70% West African ancestry. The individual may have been classified as having at least 70% West African ancestry via one or more ancestry identification methods, such as by one or more of SNP analysis, Y chromosome testing, Mitochondrial DNA testing, genealogy research, and/or public and/or church record analysis. In specific cases, the SNP analysis comprises analysis at rs6909271, rs1129038, rs1426654, rs1572396, rs16891982, rs2165139, rs3768641, rs7689609, rs2660769, rs2814778, rs7810554, rs587364, rs10264353, rs1871534, rs2439522, rs11073967, rs1540979, rs5025718, rs1931059, rs7687935, rs2065982, rs424436, rs992864, rs10908316, rs6446975, rs9290363, rs1867024, rs12714168, rs218867, rs6695965, rs794672, rs1443985, rs2458640, rs4727700, rs2021782, rs2332031, rs1557519, rs2384319, rs901304, rs10954631, rs567442, rs10032047, rs12347078, rs13385952, rs13108157, rs1881244, rs533571, rs1638567, rs2714758, rs10059859, rs6748661, rs7784684, rs6604611, rs6576989, rs4513684, rs9311121, rs17035850, rs855833, rs11124405, rs260714, rs6829588, rs7662047, rs12489482, rs2791966, rs6601288, rs1439013, rs6459548, rs11714866, rs2502342, rs6698938, rs2197896, rs10257477, rs11713766, rs6772085, rs7657799, rs2497150, rs1341567, rs6930928, rs300152, rs4478653, rs710232, rs951954, rs12074150, rs2470644, rs35395, rs463240, rs730570, rs596985, rs814597, rs6439896, rs3094537, rs6437783, rs6485600, rs1551765, rs4936512, rs13069719, rs11778591, rs7504, rs883399, rs2065160, rs10748592, rs2293048, rs1648180, rs9937955, and/or rs2274533. In specific embodiments, the individual is African-American, Jamaican-American, Haitian-American, and/or Black of any ancestral origin.

Any methods encompassed herein may comprise the step of testing for presence or level of prostate significant antigen (PSA) and/or having a digital rectal exam. In specific cases, the individual has an elevated level of (PSA) and/or a positive digital rectal exam screen compared to a standard or control.

In any method encompassed herein, the biomarkers may comprise one or more of N-Acetyl Aspartate, Glutamine, Sarcosine, Succinate, Malate, and/or a combination thereof. In some cases, the biomarkers comprise, consist essentially of, or consist of Methionine, Homocysteine, Glutamic acid, Inosine, N-Acetyl Aspartate, Glutamine, Sarcosine, Succinate, and/or Malate. In specific embodiments, the biomarkers comprise, consist essentially of, or consist of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, Succinate, N-Acetyl Aspartate, 2-Hydroxy Glutarate, Malate and/or Fumarate. In some embodiments, the biomarkers comprise, consist essentially of, or consist of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, Succinate, N-Acetyl Aspartate, 2-Hydroxy Glutarate, Malate, Homocysteine, Glutathione, Ketoglutarate, Homocysteine, Carnitine, Cholic Acid, Deoxycholic Acid, Testosterone, 5-dihydrotestosterone, Estrone, Estradiol, Progesterone, 25-hydroxyvitamin D3, and/or Fumarate.

In any method encompassed herein, the biomarkers may comprise one or more of N-Acetyl Aspartate, Glutamine, Sarcosine, Succinate, Malate, Methionine, Homocysteine, Glutamic acid, Inosine, Melatonin, gamma-Aminobutyric acid, Isoleucine, Adenosine, Putrescine, Arginine, Ornithine, Homocysteine, Valine, Kynurenine, Proline, Kynurenic acid, reduced Glutathione, Pyruvate, Lactate, alpha-Ketoglutarate, 2-Hydroxy Glutarate, Malate, Fumarate, Glutathione, Ketoglutarate, Carnitine, Cholic Acid, Deoxycholic Acid, Testosterone, Estrone, Estradiol, Progesterone, and 25-hydroxyvitamin D3.

Any methods encompassed herein may further comprise the step of obtaining a sample from the individual, such as a sample being any one or more of biopsy tissue, urine, plasma, and/or serum. The step of obtaining a sample may comprise or may further comprise a prostate screening exam. Any method of the disclosure may comprise the individual having a prostate screening exam, which may be a digital rectal exam and/or PSA test. In specific examples, the individual has a PSA level is between the range of 2.5-10 ng/mL. In specific embodiments, the prostate screening exam further comprises one or more of the steps of taking one or more of prostate biopsies, prostate ultrasound, MRI fusion, imaging, Prostate Health Index (PHI), kallikrein test, blood test, prostate cancer antigen 3 (PCA3) test, and/or epigenetic test. In certain aspects, the step of obtaining a sample further comprises determining the grade and/or aggressiveness of the cancer. In such cases, determining the grade of cancer may further comprise assigning a Gleason score, Gleason sum, Grade Group, and/or genomic testing. In specific embodiments, the Gleason score is 1, 2, 3, 4, 5, 6, 7, 8, 9, or 10; in some cases. the Gleason score indicates the group of well-differentiated or low-grade, moderately-differentiated or intermediate-grade, or poorly-differentiated or high-grade cancer.

Any method encompassed by the disclosure may comprise a step of obtaining a sample that further comprises determining the stage and/or spread of the cancer. The spread of the cancer may include one or more of metastasis to the bones, lungs, lymph nodes, liver, brain, adrenal glands, breasts, eyes, kidneys, muscles, pancreas, salivary glands, spleen, stomach, colon, testicles, anus, skin, esophagus, intestine, and/or blood. In specific embodiments, the step of determining the stage and/or spread of cancer further comprises one or more of a bone scan, ultrasound, computerized tomography (CT) scan, magnetic resonance imaging (MRI), positron emission tomography (PET) scan, prostate-specific membrane antigen (PSMA) test, advanced ultra-high-field scanning technology.

In particular embodiments, it is determined that the individual in need of treatment is at increased risk for prostate cancer, and the increased risk for prostate cancer may be associated with one or more of a family history of prostate cancer, change in one or more marker levels, family cancer syndrome, family history of breast, ovarian, colon, and/or prostate cancer, age, chemical exposure, Agent Orange exposure, obesity, regular consumption of high-fat foods and/or processed carbohydrates, sedentary lifestyle, genetic factors (presence or particular level of RNASEL, BRCA1, BRCA2, MSH2, MLH1, HOXB13, HPXC, CAPB, ATM, FANCA, HPC1, and/or HPC2), enlarged prostate, and/or prostatic intraepithelial neoplasia. In specific embodiments, a change in marker level is detected by one or more of the methods of mass spectrometry, chromatography, high-performance liquid chromatography, spectroscopy, ELISA, or immunoassay.

In any embodiments of the methods encompassed herein the method further comprises the step of measuring or predicting the cancer status of an individual. The step of measuring or predicting cancer status may further comprise the use of one or more of the prostate specific antigen score, Gleason score, and/or body mass index. The measurement or prediction of cancer status may be made by a machine learning classifier. The machine learning classifier (such as a random forest) may have been trained using biomarkers levels obtained from individuals with prostate cancer and control individuals that do not have cancer.

In some embodiments of any method of the disclosure, the step of treating the individual comprises one or more of active surveillance, surgery, cancer treatment, and/or one or more cancer treatments. The cancer treatment may comprise one or more of surgery, radiation, proton therapy, hormone therapy, chemotherapy, immunotherapy, bisphosphate therapy, cryotherapy, ultrasound, and/or palliative care. In specific cases, the cancer treatment comprises continuing the therapy, ceasing therapy, or changing the method of therapy.

In certain embodiments, there is a method of measuring or determining the grade and/or aggressiveness of a cancer comprising identifying a change in the level of one or more biomarkers. The biomarkers may comprise, consist of, or consist essentially of Methionine, Homocysteine, Glutamic acid, Inosine, and/or a combination thereof. The biomarkers may comprise, consist of, or consist essentially of Methionine, Homocysteine, Glutamic acid, Ornithine, Inosine, N-Acetyl Aspartate, Glutamine, Sarcosine, Succinate, Malate, and/or a combination thereof. The biomarkers may comprise, consist of, or consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Ornithine, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate, Fumarate, or a combination thereof. In some cases, the method further comprises measuring or determining the grade and/or aggressiveness of the cancer by analyzing a sample from an individual.

In one embodiment, there is a method of determining the stage and/or spread of the cancer comprising identifying a change in the level of certain one or more biomarkers, and the one or more biomarkers may comprise, consist of, or consist essentially of Methionine, Homocysteine, Glutamic acid, Ornithine, Inosine, and/or a combination thereof. The biomarkers may comprise, consist of, or consist essentially of Methionine, Homocysteine, Glutamic acid, Ornithine, Inosine, N-Acetyl Aspartate, Glutamine, Sarcosine, Succinate, Malate, and/or a combination thereof. The biomarkers may comprise, consist of, or consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate, Fumarate, and/or a combination thereof. In some embodiments, the method further comprises determining the stage and/or spread of the cancer by analyzing a sample from an individual.

In one embodiment, there is a method of assessing or measuring or predicting the cancer status of an individual comprising identifying a change in the level of one or more biomarkers in the individual. The biomarkers may comprise, consist of, or consist essentially of Methionine, Homocysteine, Glutamic acid, Ornithine, Inosine, and/or a combination thereof. The biomarkers may comprise, consist of, or consist essentially of Methionine, Homocysteine, Glutamic acid, Inosine, N-Acetyl Aspartate, Glutamine, Sarcosine, Succinate, Malate, or a combination thereof. The biomarkers may comprise, consist of, or consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate, Fumarate, or a combination thereof. In some embodiments, method further comprises determining the stage and/or spread of the cancer by analyzing a sample from an individual. Any cancer referred to herein may be indolent or aggressive.

In one embodiment, there is a method of treating an individual in need thereof comprising the steps of detecting a change in the level of a combination of one or more biomarkers and treating the individual in need thereof (e.g., a change in the level of any metabolite referred to herein at least 2-fold) with at least one cancer therapy.

In a certain embodiment, there is a method of identifying a subject for treatment of prostate cancer comprising the step of identifying a change in the level of a combination of biomarkers comprising, consisting of, or consisting essentially of the metabolites Methionine, Homocysteine, Glutamic acid, Ornithine, Inosine, or a combination thereof from the sample of the individual, measuring or determining or predicting cancer status of the individual, and determining treatment for the individual.

In some embodiments, there is a kit comprising any one or more compositions encompassed herein, said composition(s) housed in a suitable container.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A-1D illustrate performance of a set of models for prediction of prostate cancer status using specific biomarker sets. The bigger panel contained 9 metabolites (including Methionine, Homocysteine, Glutamic acid, Inosine, N-Acetyl Aspartate, Glutamine, Sarcosine, Succinate, and Malate) and the smaller panel contained 4 metabolites (including Methionine, Homocysteine, Glutamic acid, and Inosine). The bigger and smaller panels containing PSA were used in a logistic regression classification model with a 5-fold cross validation to calibrate the performance of the model. In presence of PSA, the bigger panel had a cross-validated Area under the Curve (AUC) of 0.71 (FIG. 1A), while the smaller panel had an AUC of 0.81 (FIG. 1B). PSA as a single covariate had a cross-validated AUC of only 0.63 (FIG. 1C). The smaller panel without PSA gave an improved cross-validated AUC of 0.93 (FIG. 1D).

FIG. 2 provides Receiver Operator Curve (ROC) using combined levels of five metabolites to detect prostate cancer (positive biopsy) in African American men. The Area Under the Curve (AUC) for detection of prostate cancer is 0.83.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the disclosure, for example. Some embodiments of the disclosure may consist of or consist essentially of one or more elements, method steps, and/or methods of the disclosure. It is contemplated that any method or composition described herein can be implemented with respect to any other method or composition described herein. Embodiments discussed in the context of methods and/or compositions of the disclosure may be employed with respect to any other method or composition described herein. Thus, an embodiment pertaining to one method or composition may be applied to other methods and compositions of the disclosure as well.

Embodiments of the present disclosure may suffice to largely replace the need for prostate biopsy in the clinical setting. The current method for diagnosing prostate cancer typically includes screening mechanisms such as a blood test for prostate-specific antigen (PSA) and/or digital rectal exam. If either screen indicates presence of prostate cancer, a prostate biopsy is performed, in which tissue at several sites on the prostate is removed with a hollow needle. Biopsies are examined using a microscope for the presence of cancer cells, and possible results include positive for cancer, negative for cancer, and suspicious, which indicates abnormal cells that are not necessarily cancer. Prostate biopsies are associated with risks including bleeding, including blood in semen and/or urine, difficulty urinating, soreness, and/or urinary tract infection. Subjects may also feel physical, social, or emotional discomfort before, during, and/or after the procedure. High PSA scores are not always indicative of prostate cancer, leading to a high incidence of unnecessary biopsies. Many patients who undergo biopsy do not have prostate cancer. Once established in the clinic, the method presented herein allows for less invasive testing, increased willingness among subjects to participate in prostate cancer screening or testing, reduction of the financial burden on individuals as well as the healthcare system, and reduction of the number of unnecessary biopsies.

Embodiments of the disclosure include systems and methods related to classification, diagnosis, or treatment of prostate cancer in an individual in need thereof. The individual is a human, in particular aspects. In some embodiments, the human is from one or more races. Examples of races of humans include Black, White, Caucasian, Asian, Hispanic and/or Latino/a, Indigenous, North African, Middle Eastern, Native Hawaiian and/or Pacific Islander, Alaskan Native and/or American Indian, Indigenous Australian, and/or two or more of these races. In some embodiments, the human of any of one or more races is comprised of one more ancestries. Examples of ancestries include West African, East African, North African, Caribbean, Western European, Eastern European, Mexican, Puerto Rican, Irish, English, American, Scandinavian, East Asian, Asian Indian, Filipino, and/or Australian. In particular embodiments, the individual is a Black and/or African American individual of West African ancestry. Most self-reported African American and/or Black individuals from the United States of America have approximately 80% West African ancestry [11]. In particular embodiments, the individual's race is identified through self-reporting, SNP analysis, Y chromosome testing, Mitochondrial DNA testing, genealogy research, and/or public and/or church record analysis. In particular embodiments, the individual is an African American or Black individual with at least 70% West African ancestry verified through SNP analysis.

In some embodiments, a method of ancestry identification is used to validate a self-report of West African ancestry. In other embodiments, the method of ancestry identification is used when an individual does not know their ancestry. In at least some cases, methods of the disclosure related to classification, diagnosis, or treatment of prostate cancer are performed when the ancestry of the individual is unknown or unverified. In some embodiments, the method of ancestry identification is SNP analysis. In some embodiments, the method of SNP analysis comprises the steps of identifying and validating single nucleotide polymorphisms (SNPs) appropriate for appraising continental ancestry in admixed populations, and creating and genotyping a panel of SNPs. The method may further comprise one or more of the following: isolating DNA from a subject in need of ancestry typing, amplifying the DNA by polymerase chain reaction (PCR), treating the PCR with shrimp alkaline phosphatase enzyme, performing a post-PCR single-base extension reaction, diluting the PCR, spotting the reaction to a microarray, scanning by MALDI-TOF mass spectrometry, and calling individual SNP genotype from allele-specific peaks according to expected masses [11]. In some embodiments, the SNP analysis comprises rs6909271, rs1129038, rs1426654, rs1572396, rs16891982, rs2165139, rs3768641, rs7689609, rs2660769, rs2814778, rs7810554, rs587364, rs10264353, rs1871534, rs2439522, rs11073967, rs1540979, rs5025718, rs1931059, rs7687935, rs2065982, rs424436, rs992864, rs10908316, rs6446975, rs9290363, rs1867024, rs12714168, rs218867, rs6695965, rs794672, rs1443985, rs2458640, rs4727700, rs2021782, rs2332031, rs1557519, rs2384319, rs901304, rs10954631, rs567442, rs10032047, rs12347078, rs13385952, rs13108157, rs1881244, rs533571, rs1638567, rs2714758, rs10059859, rs6748661, rs7784684, rs6604611, rs6576989, rs4513684, rs9311121, rs17035850, rs855833, rs11124405, rs260714, rs6829588, rs7662047, rs12489482, rs2791966, rs6601288, rs1439013, rs6459548, rs11714866, rs2502342, rs6698938, rs2197896, rs10257477, rs11713766, rs6772085, rs7657799, rs2497150, rs1341567, rs6930928, rs300152, rs4478653, rs710232, rs951954, rs12074150, rs2470644, rs35395, rs463240, rs730570, rs596985, rs814597, rs6439896, rs3094537, rs6437783, rs6485600, rs1551765, rs4936512, rs13069719, rs11778591, rs7504, rs883399, rs2065160, rs10748592, rs2293048, rs1648180, rs9937955, and/or rs2274533. In some embodiments, the panel of SNPs is genotyped by the Sequenom MassARRAY genotyping platform with iPLEX chemistry. In some embodiments, the reaction is spotted onto a Sequenom SpectroCHIP microarray. In some embodiments, individual SNP genotype calls are made with Sequenom TYPER software.

I. Metabolic Biomarker Panels

Metabolic biomarkers for identifying individuals for prostate biopsy and/or cancer treatment, and/or predicting cancer status of an individual are provided herein. In one embodiment, the method of treating an individual for prostate cancer comprises the step of administering an effective amount of treatment to an individual in need thereof when the individual has a change in the level of one or more biomarkers comprising, consisting of, or consisting essentially of Methionine, Homocysteine, Glutamic acid, Ornithine, Inosine, and/or a combination thereof. In other embodiments, the biomarkers further comprise, consist of, or consist essentially of one or more of N-Acetyl Aspartate, Glutamine, Sarcosine, Succinate, Malate, and/or a combination thereof. In other embodiments, the biomarkers comprise, consist of, or consist essentially of Methionine, Homocysteine, Glutamic acid, Inosine, N-Acetyl Aspartate, Glutamine, Sarcosine, Succinate, Malate, and/or a combination thereof. In other embodiments, the biomarkers comprise, consist of, or consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate and Fumarate. In some embodiments, the biomarkers comprise, consist of, or consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, Succinate, N-Acetyl Aspartate, 2-Hydroxy Glutarate, Malate, Homocysteine, Glutathione, Ketoglutarate, Homocysteine, Carnitine, Cholic Acid, Deoxycholic Acid, Testosterone, 5-dihydrotestosterone, Estrone, Estradiol, Progesterone, 25-hydroxyvitamin D3, and/or Fumarate. In some embodiments, the composition of metabolites comprises, consists of, or consists essentially of Melatonin, Aminobutyric Acid, Isoleucine, Adenosine, Methionine, Homocysteine, Valine, Kynurenine, Inosine, Arginine, Ornithine, Proline, Sarcosine, Putrescine, Glutathione, Glutamic acid, Glutamine, Kynurenic acid, Pyruvate, Lactate, Ketoglutarate, Acetyl Aspartic acid, Malate, 2HG, Succinate, and/or Fumarate. In other embodiments, the metabolites additionally comprise Carnitine, Cholic acid, Deoxycholic acid, Testosterone, 5-dihydrotestosterone, Estrone, Estradiol, Progesterone, and/or 25-hydroxyvitamin D3. In certain embodiments, the biomarkers include all of the above-listed metabolites and, in specific cases, no other metabolites other than those listed. In a specific embodiment, the biomarkers include all of the above-listed metabolites and 1, 2, 3, 4, 5, 6, 7, 8, 9, or 10 or more additional metabolites. In specific embodiments, the biomarkers further comprise biomarkers not listed. In some embodiments, the biomarkers lack Homocysteine and/or Inosine. In other embodiments, the biomarkers may or may not include a ratio of two or more biomarkers.

In some embodiments, the method comprises taking a sample from an individual. In some aspects, the biomarkers are present in the individual in particular tissues and/or fluids at particular levels. In some embodiments, the tissues and/or fluids include one or more of prostate tissue, blood, urine, and/or plasma. In some embodiments, the method comprises measuring biomarkers from the sample taken from the individual.

II. Methods for Using Metabolic Biomarkers

A. Methods for Identifying Subjects for Biopsy and/or Cancer Treatment

Embodiments of the disclosure include systems and methods related to classification, diagnosis, or treatment of prostate cancer in an individual in need thereof. The individual is a human. In some embodiments, the human is comprised of one or more races. In some embodiments, race is defined as a group of humans identified based on shared ancestry and common physical traits. Examples of races of humans include Black, White, Caucasian, Asian, Hispanic and/or Latino/a, Indigenous, North African, Middle Eastern, Native Hawaiian and/or Pacific Islander, Alaskan Native and/or American Indian, Indigenous Australian, and/or two or more races. In some embodiments, the human of any of one or more races is comprised of one more ancestries. In some embodiments, ancestry is defined as one's family or ethnic decent. Examples of ancestries include West African, East African, North African, Caribbean, Western European, Eastern European, Mexican, Puerto Rican, Irish, English, American, Scandinavian, East Asian, Asian Indian, Filipino, and/or Australian. In some embodiments, the individual is comprised of one or more ethnicities. In some embodiments, ethnicity is defined as a social group sharing national or cultural traditions. Examples of ethnicities include African American and/or Black, African, Caribbean, American, Alaska Native, Native American and/or Indigenous, Native Hawaiian and/or Pacific Islander, Hispanic and/or Latino/a, Chinese, Indian, Arab, Scandinavian, English/Welsh/Irish and/or one or more ethnicities. In particular embodiments, the individual is a Black and/or African American individual of Black and/or African American ethnicity and West African ancestry. Most self-reported African American and/or Black individuals from the United States of America have approximately 80% West African ancestry [11]. In particular embodiments, the individual's race is identified through self-reporting, SNP analysis, Y chromosome testing, Mitochondrial DNA testing, genealogy research, and/or public and/or church record analysis. In preferred embodiments, the individual is an African American or Black individual with at least 70% West African ancestry verified through SNP analysis.

In some embodiments, the composition of metabolites comprises, consists of, or consists essentially of Melatonin, Aminobutyric Acid, Isoleucine, Adenosine, Methionine, Homocysteine, Valine, Kynurenine, Inosine, Arginine, Ornithine, Proline, Sarcosine, Putrescine, Glutathione, Glutamic acid, Glutamine, Kynurenic acid, Pyruvate, Lactate, Ketoglutarate, Acetyl Aspartic acid, Malate, 2HG, Succinate, and/or Fumarate. In other embodiments, the metabolites additionally comprise Carnitine, Cholic acid, Deoxycholic acid, Testosterone, 5-dihydrotestosterone, Estrone, Estradiol, Progesterone, and/or 25-hydroxyvitamin D3.

Methods of the disclosure include determination of specific levels of one or more metabolites that are indicative of whether or not the individual has prostate cancer, is susceptible to prostate cancer (including at a level greater than the general male population), whether or not additional tests for prostate cancer or risk need to be performed, whether or not the individual needs a biopsy, the stage of prostate cancer, the presence of metastasis of prostate cancer, or a combination thereof. Such levels are determined from one or more samples taken from the desired individual. In a given sample or samples from an individual in need of having the level of one or more metabolites determined, there may be a range or threshold in which the level is an indicator of one of the aforementioned conditions. The following addresses such examples of ranges. In some aspects, the concentration of Melatonin ranges from 0.1-2, 0.1-1.75, 0.1-1.5, 0.1-1.25, 0.1-1, 0.5-2, 0.5-1.75, 0.5-1.5, 0.5-1, 1-2, 1-1.75, 1-1.5, or 1-1.25 μM. In some embodiments, the concentration of Aminobutyric acid ranges from 0.1-1000, 0.1-750, 0.1-500, 0.1-100, 1-1000, 1-750, 1-500, 1-250, 1-100, 100-1000, 100-750, 100-500, 100-250, 500-1000, or 500-750 μM. In some embodiments, the concentration of Isoleucine ranges from 1-1500, 1-1000, 1-500, 1-250, 1-100, 100-1500, 100-1000, 100-500, 500-1500, 500-1000, or 1000-1500 μM. In some embodiments, the concentration of Adenosine ranges from 0-150, 0-125, 0-100, 0-75, or 0-50, 10-150, 10-125, 10-100, 10-50, 50-150, 50-125, 50-100, 50-75, 100-150, or 100-125 μM. In some embodiments, the concentration of Methionine ranges from 0-500, 0-400, 0-200, 0-100, 50-500, 50-400, 50-300, 50-200, 50-100, 100-500, 100-400, 100-300, 100-200, 200-500, 200-400, 200-300, 300-500, or 300-400 μM. In some embodiments, the concentration of Homocysteine ranges from 0-50, 0-25, 0-15, 0-10, 0-5, 10-50, 10-25, 10-15, 15-15-25, or 25-50 μM. In some embodiments, the concentration of Valine ranges from 0.1-500, 0.1-300, 0.1-200, 0.1-100, 1-500, 1-400, 1-300, 1-200, 1-100, 50-500, 50-400, 50-300, 50-100, 100-400, 100-300, 100-200, 200-500, 200-400, 200-300, 300-500, 300-500, or 400-500 μM. In some embodiments, the concentration of Kynurenine ranges from 0.1-10, 0.1-5, or 0.1-1 μM. In some embodiments, the concentration of Inosine ranges from 0.1-10, 0.1-2.5, 0.1-1, 1-10, 1-5, 1-2.5, 1-2, 2-10, 2-5, 2-2.5, or 5-10 μM. In some embodiments, the concentration of Arginine ranges from 0-150, 0-125, 0-100, 0-50, or 0-25, 1-150, 1-125, 1-100, 1-50, 1-25, 10-150, 10-125, 10-100, 10-50, 10-25, 50-150, 50-100, 50-75, 100-150, or 100-125 μM. In some embodiments, the concentration of Ornithine ranges from 0.1-200, 0.1-150, 0.1-50, 1-200, 1-150, 1-100, 1-50, 1-10, 10-200, 10-150, 10-100, 10-50, 50-200, 50-150, 50-100, 100-200, 100-150, or 150-200 μM. In some embodiments, the concentration of Proline ranges from 1-400, 1-300, 1-200, 1-100, 1-50, 10-400, 10-300, 10-200, 10-100, 50-400, 50-200, 50-100, 100-400, 100-300, 100-200, 200-400, 200-300, or 300-400 μM. In some embodiments, the concentration of Sarcosine ranges from 1-400, 1-300, 1-200, 1-100, 1-50, 10-400, 10-300, 10-200, 10-100, 50-400, 50-300, 50-200, 50-100, 100-400, 100-300, 100-200, 200-400, 200-300, or 300-400 μM. In some embodiments, the concentration of Putrescine ranges from 0.1-250, 0.1-200, 0.1-150, 0.1-100, 1-250, 1-200, 1-150, 1-100, 1-50, 1-10, 10-250, 10-200, 10-100, 10-50, 50-250, 50-200, 50-150, 50-100, 100-250, 100-200, 100-150, or 200-250 μM. In some embodiments, the concentration of Glutathione ranges from 0.1-600, 0.1-500, 0.1-400, 0.1-300, 0.1-200, 0.1-100, 0.1-50, 1-600, 1-500, 1-400, 1-300, 1-200, 1-100, 1-50, 1-10, 50-600, 50-500, 50-400, 50-300, 50-200, 50-100, 100-600, 100-500, 100-400, 100-300, 100-200, 200-600, 200-500, 200-400, 200-300, 300-600, 300-500, 300-400, 400-600, 400-500, or 500-600 μM. In some embodiments, the concentration of Glutamic acid ranges from 0.1-20, 0.1-10, 0.1-5, 0.1-1, 1-20, 1-10, 1-5, 1-2.5, 5-20, 5-15 5-10, 10-20, 10-15, or 15-20 μM. In some embodiments, the concentration of Glutamine ranges from 1-1500, 1-1250, 1-1000, 1-750, 1-500, 50-1500, 50-1250, 50-1000, 50-750, 50-500, 50-250, 50-100, 100-1500, 100-1250, 100-1000, 100-750, 100-500, 100-250, 500-1500, 500-1250, 500-750, 1000-1500, 1000-1250, or 1250-1500 μM. In some embodiments the concentration of Kynurenic acid ranges from 0-3, 0-2.5, 0-2, 0-1.5, 0-1, 1-3, 1-2.5, 1-2, 1-1.5, 1.5-3, 1.5-2, 2-3, 2-2.5, or 2.5-3 μM. In some embodiments, the concentration of Pyruvate ranges from 0-150, 0-100, 0-50, 0-25, 0-10, 10-150, 10-50, 10-25, 25-150, 25-100, 25-50, 50-150, 50-100, or 100-150 μM. In some embodiments, the concentration of Lactate ranges from 40-5000, 40-4000, 40-3000, 40-2000, 100-5000, 100-4000, 100-3000, 100-2000, 100-1000, 500-5000, 500-4000, 500-3000, 500-2000, 500-1000, 1000-5000, 1000-4000, 1000-3000, 1000-2000, 2000-5000, 2000-4000, 2000-3000, 3000-5000, 3000-4000, or 4000-5000 μM. In some embodiments, the concentration of Ketoglutarate ranges from 0-10, 0-8, 0-6, 0-4 0-2, 1-10, 1-8, 1-6, 1-4, 1-2, 2-10, 2-8, 2-6, 2-4, 4-10, 4-8, 4-6, 6-10, 6-8, or 8-10 μM. In some embodiments, the concentration of Acetyl Aspartic acid ranges from 0.01-3, 0.01-2.5, 0.01-2, 0.01-1.5, or 0.01-1, 0.1-3, 0.1-2.5, 0.1-2, 0.1-1.5, 0.1-1, 0.1-0.5, 1-3, 1-2.5, 1-2, 1-1.5, 2-3, 2-2.5, or 2.5-3 μM. In some embodiments, the concentration of Malate ranges from 0.1-3, 0.1-2.5, 0.1-2, 0.1-1.5, 0.1-1, 0.1-0.5, 1-3, 1-2.5, 1-2, 1-1.5, 2-3, 2-2.5, or 2.5-3 μM. In some embodiments, the concentration of 2HG ranges from 0-1, 0-0.8, 0-0.6, 0-0.4, 0-0.2, 0.1-1, 0.1-0.8, 0.1-0.6, 0.1-0.4, 0.1-0.2, 0.5-1, 0.5-0.8, 0.5-0.6, or μM. In some embodiments, the concentration of Succinate ranges from 0-3, 0-2.5, 0-2, 0-1.5, 0-1, 1-3, 1-2.5, 1-2, 1-1.5, 1.5-3, 1.5-2, 2-3, 2-2.5, or 2.5-3 μM. In some embodiments, the concentration of Fumarate ranges from 3-6, 3-5.5, 3-5, 3-4.5, 3-4, 4-6, 4-5.5, 4-5, 4-4.5, 5-6, 5-μM. In some embodiments, the concentration of Homocysteine ranges from 0-15, 0-10, 0-5, 1-15, 1-10, 1-5, 5-10, 5-15, or 10-15. In some embodiments, the concentration of any one of Carnitine, Cholic acid, Deoxycholic acid, Testosterone, 5-dihydrotestosterone, Estrone, Estradiol, Progesterone, or 25-hydroxyvitamin D3 ranges from 0-5000, 1-5000, 1-4000, 1-3000, 1-2000, 1-1000, 100-5000, 100-4000, 100-3000, 1-2000, 1-1000, 100-5000, 100-4000, 100-3000, 100-2000, 100-1000, 1000-5000, 1000-4000, 1000-3000, 1000-2000, 2000-5000, 2000-4000, 2000-3000, 3000-5000, 3000-4000, 4000-5000 μM. In some embodiments, the method further comprises one or more of the steps of determining a measurement for one or more metabolites, combining the information gained from the measurement of one or more metabolites, and making a decision based on information gained from the metabolite measurements. In particular embodiments, the metabolite levels are measured relative to levels of one or more of another metabolite.

In particular embodiments, the level of one or more metabolites from a sample from an individual is at a level that is indicative of having prostate cancer or being at high risk for having prostate cancer. In specific aspects, the level of certain metabolites for being very confident (over 90% prob) for Positive biopsy status is as follows (all concentrations in μM): methionine >2.10; homocysteine >0.02; glutamic Acid >5; and inosine >0.02.

Methods for using the disclosed biomarkers and methods to identify an individual for prostate cancer testing and/or treatment are included herein. The individual identified for prostate cancer testing and/or treatment may have prostate cancer, or may have an increased risk of developing cancer relative to the general population. The individual may present with an elevated PSA score or PSA score of between 2.5 and 10 ng/mL and/or present with a positive digital rectal exam. In some embodiments, the method further comprises the steps of acquiring a sample containing metabolites from the individual in need thereof, testing additional clinical presentations such PSA score and/or body mass index, and/or detecting quantities of biomarkers selected from one of the disclosed sets of biomarkers from the sample. In some embodiments, the sample is one or more of blood, plasma, serum, and/or urine. In some embodiments, the step of obtaining a sample further comprises the individual having a prostate screening exam. In some embodiments, the prostate screening exam is a digital rectal exam and/or PSA test. In other embodiments, the prostate screening exam additionally comprises one or more of the steps of taking one or more of prostate biopsies, prostate ultrasound, MRI fusion, imaging, Prostate Health Index (PHI), 4kscore test, kallikrein test, blood test, prostate cancer antigen 3 (PCA3) test, epigenetic testing, and/or ConfirmMDx test. In some embodiments, the sample is obtained from the individual at the same time as the prostate screening exam. In other embodiments, the sample is obtained from the individual at a different time from the prostate screening exam.

In some embodiments, the individual is at increased risk for prostate cancer. In some embodiments, the increased risk is associated to one or more of a family history of prostate cancer, family cancer syndrome, family history of breast, ovarian, colon, and/or prostate cancer, age, chemical exposure, Agent Orange exposure, obesity, regular consumption of high-fat foods and/or processed carbohydrates, sedentary lifestyle, genetic factors, enlarged prostate, and/or prostatic intraepithelial neoplasia. In some embodiments, genetic factors associated with an increased risk of prostate cancer include one or more of a change in expression of RNASEL, BRCA1, BRCA2, MSH2, MLH1, HOXB13, HPXC, CAPB, ATM, FANCA, HPC1, and/or HPC2. In other embodiments, the genetic factors associated with an increased risk of prostate cancer include a change in expression of other genes not listed.

In some embodiments, the method comprises treating an individual for prostate cancer comprising the step of administering an effective amount of one or more treatments to an individual in need thereof when the individual has a change in the level of biomarkers comprising Methionine, Homocysteine, Glutamic acid, Inosine, and/or a combination thereof. In some embodiments, the method comprises treating an individual for prostate cancer comprising the step of administering an effective amount of one or more treatments to an individual in need thereof when the individual has a change in the level of biomarkers comprising Melatonin, Aminobutyric Acid, Isoleucine, Adenosine, Methionine, Homocysteine, Valine, Kynurenine, Inosine, Arginine, Ornithine, Proline, Sarcosine, Putrescine, Glutathione, Glutamic acid, Glutamine, Kynurenic acid, Pyruvate, Lactate, Ketoglutarate, Acetyl Aspartic acid, Malate, 2HG, Succinate, Fumarate, and/or a combination thereof. In other embodiments, the method comprises treating an individual for prostate cancer comprising the step of administering an effective amount of one or more treatments to an individual in need thereof when the individual has a change in the level of biomarkers comprising Carnitine, Cholic acid, Deoxycholic acid, Testosterone, 5-dihydrotestosterone, Estrone, Estradiol, Progesterone, and/or 25-hydroxyvitamin D3. In other embodiments, the method comprises determining the grade and/or aggressiveness of a cancer comprising identifying a change in the level of biomarkers. In some embodiments, the method further comprises determining the stage and/or spread of the cancer comprising identifying a change in the level of biomarkers. In some aspects, the method comprises predicting the cancer status of an individual comprising identifying a change in the level of biomarkers. In some embodiments, the method of treating an individual in need thereof comprises the steps of detecting a change in the level of a combination of biomarkers and treating the individual in need thereof with at least one cancer therapy. In some embodiments, the method comprises identifying a subject for treatment of prostate cancer comprising the step of identifying a change in the level of a combination of biomarkers comprising, consisting of, or consisting essentially of the metabolites Methionine, Homocysteine, Glutamic acid, Inosine, and/or a combination thereof from the sample of the individual, predicting cancer status of the individual, and determining treatment for the individual.

B. Methods for Detecting Levels of Metabolic Biomarkers

The biomarkers disclosed can be detected using a variety of methods. These could include mass spectrometry, chromatography, high-performance liquid chromatography, spectroscopy, ELISA, immunoassay, immunoblot analysis, radioimmunoassay, gas chromatography, enzyme assays, reverse phase protein microarrays, dot blots, microfluidics, and/or other known methods. In particular embodiments, the metabolic biomarkers are quantified using mass spectrometry.

In specific aspects, the metabolite standards and control compositions are prepared with a suitable ratio of metabolite to diluent and parsed out into suitable concentrations. Clinical samples are added to diluent in a suitable ratio, and specific metabolites with thiol or sulfide groups are derivatized. Labeled standard solution and compounds are added as standards. The supernatant aqueous extract is deproteinized using a filter, from which metabolites are captured and dried. The filtrate extract is resuspended in appropriate solvent and subjected to liquid chromatography. In a specific example, a mix of 26 metabolite standards are freshly prepared with 1:1 MeOH:H₂O at the concentration of 200 μM. 1:2 serial dilution was performed with 1:1 MeOH:H₂O to make subsequent concentration of 100, 50, 25, 12.5, 6.25, 3.125, 1.5625, 0.39, 0.1953, 0.098 μM. 10 μl of each standard solution was added to 90 μl Mass Spect Gold® Human Plasma, resulting in a final serial concentration of 20, 10, 5, 2.5, 1.25, 0.625, 0.15625, 0.078, 0.039, 0.01953, 0.0098 μM, followed by vortex for 5 min. 100 μl clinical plasma sample solutions (90 μl plasma+10 μl1:1 MeOH:H₂O) or plasma standard solutions were mixed with 10 μl of 100 mM N-methylmaleimide (NMM) to derivatize the metabolites with thiol or sulfide group by mixing for 10 min. 5 μl of 100 μM labelled standard solution was added to each tube and vortex for 10 min. This was followed by the addition of ice cold 1% trifluoroacetic acid in acetonitrile, vortex for 15 min, and centrifugation. The supernatant aqueous extract was deproteinized using a 3-KDa molecular filter and the filtrate containing the metabolites was dried under vacuum. Prior to injection, the dried extract was resuspended in an injection solvent composed of water and acetonitrile (1:4) and subjected to liquid chromatography (LC)-MS.

In a specific embodiment, a mix of 26 metabolite standards are freshly prepared with 1:1 MeOH:H₂O at the concentration of 200 μM. 1:2 serial dilution was performed with 1:1 MeOH:H₂O to make subsequent concentrations of 100, 50, 25, 12.5, 6.25, 3.125, 1.5625, 0.78, 0.1953, 0.098 μM. Five μl of each standard solution was added to 45 μl Mass Spect Gold® Human Plasma, resulting in a final serial concentration of 20, 10, 5, 2.5, 1.25, 0.625, 0.3125, 0.15625, 0.078, 0.039, 0.01953, 0.0098 μM, followed by vortex for 5 min. 50 μl clinical plasma sample solutions (45 μl plasma+5 μl1:1 MeOH:H₂O) or plasma standard solutions were mixed with 5 μl of 100 mM N-ethylmaleimide (NEM) to derivatize the metabolites with thiol or sulfide group by mixing for 10 min. 2 μl of 100 μM labelled standard solution was added to each tube and vortex for 10 min. This was followed by the addition of ice cold 1% trifluoroacetic acid in acetonitrile, vortex for 15 min, and centrifugation. The supernatant aqueous extract was deproteinized using a 3-KDa molecular filter and the filtrate containing the metabolites was dried under vacuum. Prior to injection, the dried extract was resuspended in an injection solvent composed of water and acetonitrile (1:4) and subjected to liquid chromatography (LC)-MS.

In some aspects, the mass spectrometry experimental design is adjusted to settings appropriate for the method. In some embodiments, setting and/or changing the experimental design and/or parameters includes choosing one or more of a mass spectrometer and HPLC system, coupling system between the mass spectrometer and HPLC, gas temperature, gas flow rate, capillary, nozzle voltage, number of data points collected, one or more columns, one or more buffers, incubation times, incubation temperatures, run times, run temperatures, solvent concentrations, and/or analysis software. As a specific example, suspended samples (10 μL) were injected and analyzed using a 6495 triple quadrupole mass spectrometer (Agilent Technologies®, Santa Clara, CA) coupled to a 1290 HPLC system (Agilent Technologies®, Santa Clara, CA) via MRM (18 metabolites in positive mode). The source parameters were as follows: gas temperature, 250° C.; gas flow rate, 14 L/min; nebulizer, 20 psi; sheath gas temperature, 350° C.; sheath gas flow rate, 12 L/min; capillary, 3000 V positive and 3000 V negative; nozzle voltage, 1500 V positive and 1500 V negative. Approximately 8-11 data points were acquired per detected metabolite. Samples were delivered to the mass spectrometer via normal phase chromatography using either a 4.6 mm ID×10 cm Poroshell® HILIC-Z column (Agilent®). Gradients were run from 85% buffer (B; 0.1% formic acid in acetonitrile) to 35% B from 0-3.5 min, 35% B to 2% B from 3.5-11.5 min, held in 2% B from 11.5-16.5 min, from 2% B to 85% B from 16.5-17.5 min, and held in 85% B for 7 min to re-equilibrate the column. The concentration for each metabolite was quantified using Mass Hunter® Workstation Software Quantitative Analysis Version B.07.01 software (Agilent®).

In another example, suspended samples (5 μl) were injected and analyzed using a 6495 triple quadrupole mass spectrometer (Agilent Technologies®, Santa Clara, CA) coupled to a 1290 HPLC system (Agilent Technologies®, Santa Clara, CA) via MRM (8 metabolites in negative mode). The source parameters were as follows: gas temperature, 200° C.; gas flow rate, 11 L/min; nebulizer, 40 psi; sheath gas temperature, 300° C.; sheath gas flow rate, 12 L/min; capillary, 3000 V positive and 3500 V negative; nozzle voltage, 1000 V positive and 1000 V negative. Approximately 8-11 data points were acquired per detected metabolite. Samples were delivered to the mass spectrometer via normal phase chromatography using either a 2.1 mm ID×cm Poroshell® HILIC-Z column (Agilent®). Gradients were run from 90% buffer (B; 10% of 100 mM Ammonium acetate with PH at 9 in acetonitrile) for 2 min, to 60% B from 2-12 min, 60% B to 90% B from 12-13 min, held in 90% B for 7 min to re-equilibrate the column. The concentration for each metabolite was quantified using Mass Hunter® Workstation Software Quantitative Analysis Version B.07.01 software (Agilent®).

C. Methods for Predicting an Individual's Need for Biopsy and/or Cancer Treatment

The expression level of the disclosed biomarkers of interest are quantified to determine whether the individual should undergo a prostate biopsy and/or one or more cancer treatments. In some embodiments, to build a predictive tool, quantifications of expression data of biomarkers should be calculated from individuals known to have prostate cancer and individuals known not to have prostate cancer. In some embodiments, a predictive computational model is used to determine whether an individual needs a biopsy and/or cancer treatment or does not need a biopsy and/or cancer treatment. In some embodiments, the predictive computational model is a machine learning classifier. In some embodiments, the machine learning classifier is a supervised model. In other embodiments, the machine learning classifier is an unsupervised model. In some embodiments, the supervised machine learning classifier is a Support Vector Machine, linear regression, logistic regression, naïve Bayes model, linear discriminant analysis, decision trees, k-nearest neighbor algorithm, neural network, or similarity learning model. In specific embodiments, the decision tree model is a random forest. In preferred embodiments, the random forest, is able to accurately identify whether an individual will require a biopsy and/or cancer treatment or will not require a biopsy and/or cancer treatment.

In some embodiments, the method further comprises determining the grade and/or aggressiveness of the cancer. In preferred embodiments, determining the grade or aggressiveness of the cancer comprises identifying a change in the level of any set of biomarkers referred to in the preceding. In some embodiments, the method further comprises determining the grade and/or aggressiveness of the cancer by analyzing a sample from an individual. In some embodiments of the disclosure, the cancer is indolent. In other embodiments of the disclosure, the cancer is aggressive. In some embodiments, determining the grade of the cancer further comprises one or more of assigning a Gleason score, Gleason sum, Grade Group, and/or genomic testing. In some embodiments, the Gleason score is any one of 1, 2, 3, 4, 5, 6, 7, 8, 9, or 10. In some embodiments the Gleason score indicates the group of well-differentiated or low-grade, moderately-differentiated or intermediate-grade, or poorly-differentiated or high-grade cancer. In particular embodiments, the Grade Group is one of 1, 2, 3, 4, or 5. In some embodiments, the method further comprises a step of determining the stage and/or spread of the cancer comprising identifying a change in the level of biomarkers. In preferred embodiments, determining the grade or aggressiveness of the cancer comprises identifying a change in the level of any set of biomarkers referred to in the preceding. In some embodiments, the spread of the cancer includes one or more of metastasis to the bones, lungs, lymph nodes, liver, brain, adrenal glands, breasts, endometrium, esophagus, eyes, cornea, kidneys, muscles, pancreas, salivary glands, spleen, stomach, colon, testicles, anus, skin, esophagus, intestine, and/or blood. In some embodiments, the step of determining the stage and/or spread of cancer further comprises one or more of a bone scan, ultrasound, computerized tomography (CT) scan, magnetic resonance imaging (MRI), positron emission tomography (PET) scan, prostate-specific membrane antigen (PSMA) test, and/or advanced ultra-high-field scanning technology.

In some embodiments, the method further comprises the step of predicting the cancer status of an individual. In preferred embodiments, predicting the cancer status of an individual comprises identifying a change in the level of any set of biomarkers referred to in the preceding. In some embodiments, the step of predicting cancer status further comprises the use of one or more of the PSA score, Gleason score, Gleason sum, Grade Group, and/or body mass index.

IV. Methods for Treating Cancer

In some embodiments, the model predicts that the individual should not undergo biopsy. In specific embodiments, the model predicts that the individual should not undergo biopsy but should begin an observation, ‘watchful waiting, and/or active surveillance plan. In other embodiments, the model predicts that the individual should not undergo biopsy and will not need to take any other action. In some embodiments, the model predicts that the individual will need to undergo biopsy without cancer treatment, biopsy and cancer treatment, and/or cancer treatment without biopsy. In some embodiments, the biopsy will reveal the presence of cancer cells and the need to begin one or more cancer therapies.

Prostate cancer detected in an individual using the disclosed methods and systems can be treated using any of the known methods. Methods for treating cancer can include surgery, radiation, proton therapy, hormone therapy, chemotherapy, immunotherapy, bisphosphate therapy, cryotherapy, ultrasound, palliative care, bone marrow transplant, and/or drug therapy. Standard treatments for prostate cancer include one or more of surgery, radiation, chemotherapy, and/or immunotherapy. In some embodiments, the cancer treatment comprises one or more of continuing the therapy, ceasing the therapy, and/or changing the method of therapy.

In typical cases, chemotherapy for prostate cancer comprises docetaxel, cabazitaxel, mitoxantrone, and/or estramustine. In other aspects, chemotherapy for prostate cancer comprises doxorubicin, etoposide, vinblastine, paclitaxel, vinorelbine, carboplatin, oxaliplatin, and/or cisplatin. In most cases, chemotherapy starts with docetaxel, and replaced with cabazitaxel in cases where docetaxel fails. In some embodiments, the chemotherapy is combined with steroid drugs. In other embodiments, the chemotherapy is combined with other treatments. In some embodiments, the chemotherapy is administered through one or more of an infusion, orally, central venous catheter, central venous access device, central line. In some embodiments, chemotherapy is administered in cycles. In particular embodiments, cycles are 2-3 weeks long with rest periods where no chemotherapy is administered in between each cycle. In other embodiments, cycles are shorter than 2-3 weeks or longer than 2-3 weeks.

V. Kits of the Disclosure

Any of the compositions described herein may be comprised in a kit. In a non-limiting example, a set of metabolite standards, isotope labeled metabolite standards, one or more buffers and/or solvents, one or more software packages, metabolomics library, columns, plates, lipid depletion plates, sample collection containers, derivatization agents, and/or additional agent, may be comprised in a kit. In specific embodiments, the kit comprises a subset of all metabolites referenced in the present disclosure. The kits will thus comprise, in suitable container means, a set of metabolite standards, isotope labeled metabolite standards, one or more software packages, and/or an additional agent of the present invention.

The kits may comprise a suitably aliquoted set of metabolite standards, and/or set of isotope labeled metabolite standards, and/or additional agent compositions of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. The kit may also comprise an additional container means for containing a sterile, pharmaceutically acceptable buffer and/or other diluent. However, various combinations of components may be comprised in a vial. The kits of the present invention also will typically include a means for containing the set of metabolite standards, and/or set of isotope labeled metabolite standards, additional agent, vials, and any other reagent containers in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Biomarker for Prostate Cancer Detection and Prognosis in African-American Men

African American (AA) men have 60% higher incidence and two-times greater risk of dying of prostate cancer (PCa) than European American (EA) men. AA men have early onset of the disease with rapid progression to metastasis. With the failure of Prostate Specific Antigen (PSA) alone as reliable marker for PCa detection, there is an urgent need to develop accurate biomarkers for early detection of this disease especially in AA men. Metabolites can be detected in non-invasive biofluids. A metabolite-based biomarker panel (used interchangeably with ‘set of biomarkers’ or ‘biomarkers’) for cancer is currently lacking. Metabolic profiles of AA PCa were used to build a multiplex metabolite-based biomarker panel in plasma for early detection of the disease in AA men with elevated PSA.

Absolute levels of 26 metabolites associated with AA PCa were quantified using an Agilent® 6495 liquid chromatography mass spectrometry using Single Reaction Monitoring (SRM). For each metabolite, Limit of Quantification (LLOQ), matrix effect and intra/inter-day assay variability were calculated. Calibration curve for each metabolite was generated using an isotopically labeled internal standard. Training and testing approach was employed to develop and validate the metabolic panel. Pre-biopsy plasma samples from ancestry verified AA men with elevated PSA were used to develop the biomarker panel. 81 biopsy positive and 43 biopsy negative samples were used for training. 15 biopsy positive and 7 biopsy negative samples were used for testing. Biopsy results were used as reference to determine the performance of the biomarker panel.

LLOQ for all 26 quantified metabolites was less than 5 nM. Intra/inter-day variation was all less than 20%. A total of 124 AA samples with 81 biopsy-positive and 43 biopsy-negative plasma samples were employed to train a random forest model based on 10,000 trees to identify a panel of metabolites and other covariates (e.g. PSA, ancestry, Gleason score, Body Mass Index (BMI), etc.) that strongly correlate with the biopsy outcome. This approach initially defined two marker panels, both containing PSA as a co-variate. The bigger panel contained 9 metabolites and the smaller one contained 4 metabolites. Using a variable importance measure, the bigger panel had a strong predictive power for biopsy outcome with a cross-validated error rate of 9%, which was further improved by the smaller panel. Next, both the big and the small panels containing PSA were used in a logistic regression classification model. In each case, a 5-fold cross validation was used to calibrate the performance of the model. In presence of PSA, the bigger panel had a cross-validated Area Under the Curve (AUC) of 0.71, while the smaller panel had an AUC of 0.81. PSA as a single covariate had a cross-validated AUC of only 0.63. Interestingly, the smaller panel without PSA gave an improved cross-validated AUC of 0.93. The model was used to predict the biopsy outcome of 22 individuals having elevated PSA. Importantly, this training model was able to accurately identify the biopsy status in 21/22 individuals examined.

### Example 2

### Use of the Metabolic Markers to Predict Necessity of Biopsy for an Individual at Risk for Prostate Cancer

The method is applied to an individual to determine whether a prostate biopsy is necessary. An individual at increased risk for prostate cancer presents at the clinic. The prostate specific-antigen score of the patient is found to be between 2.5 and 10 ng/mL. Metabolites from the individual's plasma, including at least Methionine, Homocysteine, Glutamic acid, Ornithine, and Inosine are quantified using the method presented herein. The metabolite quantities, along with other factors including prostate-specific antigen score, and body mass index are provided to the prediction algorithm in order to determine whether or not the individual may have cancer and should undergo a prostate biopsy.

### Example 3

### Identification of Metabolic Markers Predictive of Prostate Cancer Status and Use in Classification of an Individual's Prostate Cancer Status

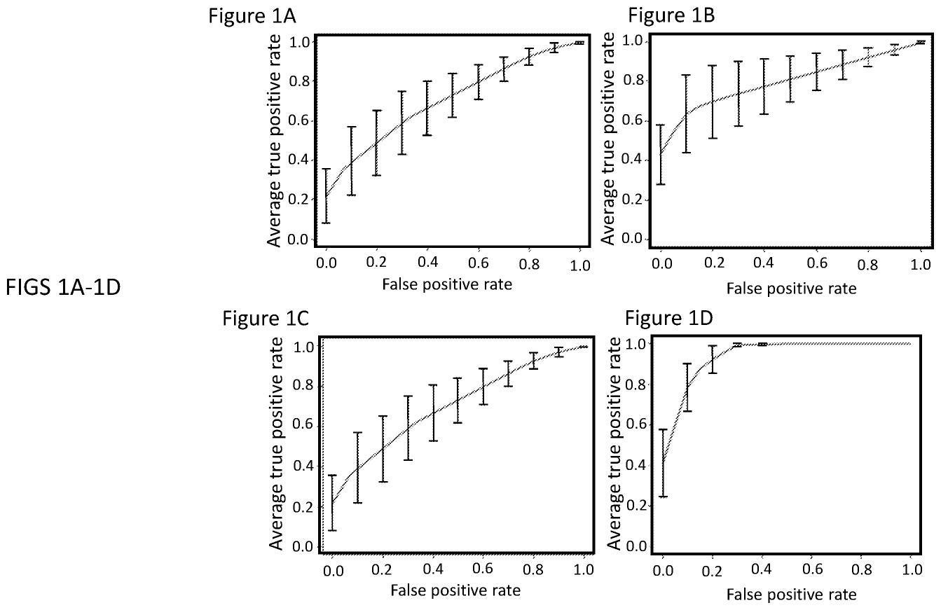

A total of 125 AA samples with 83 biopsy-positive and 42 biopsy-negative plasma samples were employed to train a random forest model based on 10,000 trees. This helped identify a panel of metabolites and other covariates (e.g. PSA, ancestry, Gleason score, Body Mass Index (BMI), etc.) that strongly correlate with the biopsy outcome. Using this approach, we initially defined two marker panels, both containing PSA as a co-variate. The bigger panel contained 9 metabolites (including Methionine, Homocysteine, Glutamic acid, Inosine, N-Acetyl Aspartate, Glutamine, Sarcosine, Succinate and Malate) and the smaller one contained 4 metabolites (including Methionine, Homocysteine, Glutamic acid and Inosine). Using a variable importance measure, the bigger panel had a strong predictive power for biopsy outcome with a cross-validated error rate of 9%, which was further improved by the smaller panel. Next, both the big and the small panels containing PSA were used in a logistic regression classification model. In each case, a 5-fold cross validation was used to calibrate the performance of the model. In presence of PSA, the bigger panel had a cross-validated Area under the Curve (AUC) of 0.71 (FIG. 1A), while the smaller panel had an AUC of 0.81 (FIG. 1B). PSA as a single covariate had a cross-validated AUC of only 0.63 (FIG. 1C). The smaller panel without PSA gave an improved cross-validated AUC of 0.93 (FIG. 1D). This model was then utilized to predict the biopsy outcome of 22 individuals having elevated PSA. Importantly, this training model was able to accurately identify the biopsy status in 21/22 individuals examined.

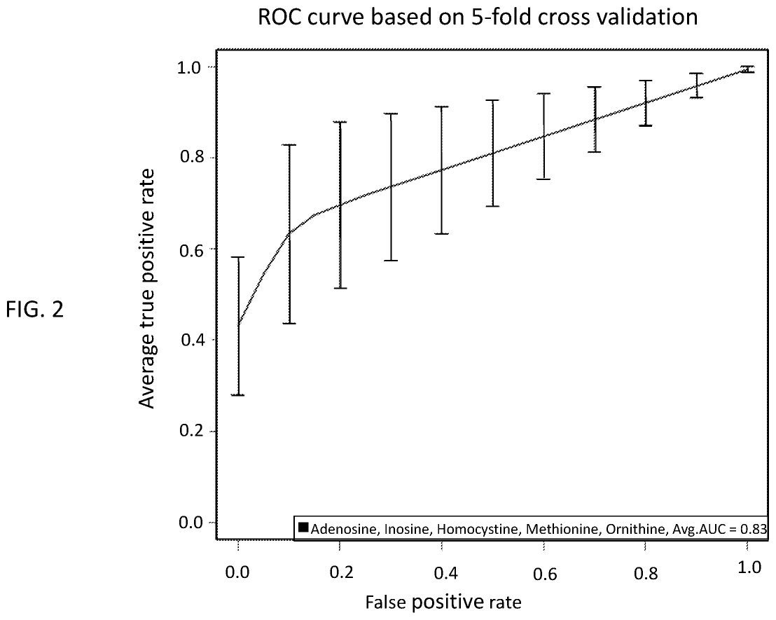

In an additional embodiment, a two stage model is trained, wherein the first stage only homocysteine is used on the training set comprising of 97 AA samples. Lower and upper cut-off values are determined. Any sample whose homocysteine value falls below or above the lower and upper cutoff values, is classified as negative and positive, respectively. If it falls in between, no decision is made and the sample is classified based on a model that comprises of the following metabolites: Adenosine, Inosine, Methionine and Ornithine. The performance of the two-stage model is assessed based on 5-fold cross validation and the average AUC value is computed (FIG. 2).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

1. Siegel, R. L., K. D. Miller, and A. Jemal, Cancer statistics, 2015. CA Cancer J Clin, 2015. 65(1): p. 5-29.
2. Lilja, H., D. Ulmert, and A. J. Vickers, Prostate-specific antigen and prostate cancer: prediction, detection and monitoring. Nat Rev Cancer, 2008. 8(4): p. 268-78.
3. Partin, A. W., et al., The use of prostate specific antigen, clinical stage and Gleason score to predict pathological stage in men with localized prostate cancer. J Urol, 1993. 150(1): p. 110-4.
4. Serefoglu, E. C., et al., How reliable is 12-core prostate biopsy procedure in the detection of prostate cancer? Can Urol Assoc J, 2013. 7(5-6): p. E293-8.
5. Xu, J., et al., Patient perspective on watchful waiting/active surveillance for localized prostate cancer. J Am Board Fam Med, 2012. 25(6): p. 763-70.
6. Massie, C. E., et al., The androgen receptor fuels prostate cancer by regulating central metabolism and biosynthesis. EMBO J, 2011. 30(13): p. 2719-33.
7. Velasco, A. M., et al., Identification and validation of novel androgen-regulated genes in prostate cancer. Endocrinology, 2004. 145(8): p. 3913-24.
8. Mitsiades, N., et al., Distinct patterns of dysregulated expression of enzymes involved in androgen synthesis and metabolism in metastatic prostate cancer tumors. Cancer Res, 2012. 72(23): p. 6142-52.
9. Kibel, A. S., Urologic Clinics of North America. Castrate-resistant prostate cancer. Preface. Urol Clin North Am, 2012. 39(4): p. xv.
10. Masson, S. and A. Bahl, Metastatic castrate-resistant prostate cancer: dawn of a new age of management. BJU Int, 2012. 110(8): p. 1110-4.
11. Hooker S E., et al., Genetic ancestry analysis reveals misclassification of commonly used cancer cell lines. Cancer Epidemiology and Prevention Biomarkers, 2019. 28(6): p. 1003-9.

What is claimed is:

1. A method of treating an individual for prostate cancer comprising the step of administering an effective amount of treatment to an individual in need thereof when the individual has a change in the level of biomarkers comprising Methionine, Homocysteine, Glutamic acid, Ornithine, Inosine, and/or a combination thereof.

2. The method of treating an individual for prostate cancer comprising the step of administering an effective amount of treatment to an individual in need thereof when the individual has a change in the level of biomarkers consisting of Methionine, Homocysteine, Glutamic acid, Ornithine, Inosine, and/or a combination thereof.

3. A method of treating an individual for prostate cancer comprising the step of administering an effective amount of treatment to an individual in need thereof when the individual has a change in the level of biomarkers consisting essentially of Methionine, Homocysteine, Glutamic acid, Ornithine, Inosine, and/or a combination thereof.

4. The method of any one of claims 1-3, wherein the individual is classified as having at least 70% West African ancestry.

5. The method of claim 4, wherein the individual has been classified as having at least 70% West African ancestry via one or more ancestry identification methods.

6. The method of claim 5, wherein the ancestry identification method is one or more of SNP analysis, Y chromosome testing, Mitochondrial DNA testing, genealogy research, and/or public and/or church record analysis.

7. The method of claim 6, wherein the SNP analysis comprises rs6909271, rs1129038, rs1426654, rs1572396, rs16891982, rs2165139, rs3768641, rs7689609, rs2660769, rs2814778, rs7810554, rs587364, rs10264353, rs1871534, rs2439522, rs11073967, rs1540979, rs5025718, rs1931059, rs7687935, rs2065982, rs424436, rs992864, rs10908316, rs6446975, rs9290363, rs1867024, rs12714168, rs218867, rs6695965, rs794672, rs1443985, rs2458640, rs4727700, rs2021782, rs2332031, rs1557519, rs2384319, rs901304, rs10954631, rs567442, rs10032047, rs12347078, rs13385952, rs13108157, rs1881244, rs533571, rs1638567, rs2714758, rs10059859, rs6748661, rs7784684, rs6604611, rs6576989, rs4513684, rs9311121, rs17035850, rs855833, rs11124405, rs260714, rs6829588, rs7662047, rs12489482, rs2791966, rs6601288, rs1439013, rs6459548, rs11714866, rs2502342, rs6698938, rs2197896, rs10257477, rs11713766, rs6772085, rs7657799, rs2497150, rs1341567, rs6930928, rs300152, rs4478653, rs710232, rs951954, rs12074150, rs2470644, rs35395, rs463240, rs730570, rs596985, rs814597, rs6439896, rs3094537, rs6437783, rs6485600, rs1551765, rs4936512, rs13069719, rs11778591, rs7504, rs883399, rs2065160, rs10748592, rs2293048, rs1648180, rs9937955, and/or rs2274533.

8. The method of any one of claims 1-3, wherein the individual is African-American, Jamaican-American, Haitian-American, and/or Black.

9. The method of any one of claims 1-8, further comprising the step of testing prostate significant antigen (PSA) and/or having a digital rectal exam.

10. The method of any one of claims 1-9, wherein the individual has an elevated level of (PSA) and/or a positive digital rectal exam screen.

11. The method of claim 1, wherein the biomarkers further comprise one or more of N-Acetyl Aspartate, Glutamine, Ornithine, Sarcosine, Succinate, Malate, and/or a combination thereof.

12. The method of claim 1, wherein the biomarkers consist essentially of Methionine, Homocysteine, Glutamic acid, Ornithine, Inosine, N-Acetyl Aspartate, Glutamine, Sarcosine, Succinate, and/or Malate.

13. The method of claim 1, wherein the biomarkers consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, Succinate, N-Acetyl Aspartate, 2-Hydroxy Glutarate, Malate and/or Fumarate.

14. The method of claim 1, wherein the biomarkers consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, Succinate, N-Acetyl Aspartate, 2-Hydroxy Glutarate, Malate, Homocysteine, Glutathione, Ketoglutarate, Homocysteine, Carnitine, Cholic Acid, Deoxycholic Acid, Testosterone, 5-dihydrotestosterone, Estrone, Estradiol, Progesterone, 25-hydroxyvitamin D3, and/or Fumarate.

15. The method of any of claims 1-14, wherein the method further comprises the step of obtaining a sample from the individual.

16. The method of claim 15, wherein the sample is any one or more of biopsy tissue, urine, plasma, and/or serum.

17. The method of claim 15, wherein the step of obtaining a sample further comprises a prostate screening exam.

18. The method of claim 15, wherein the method further comprises the individual having a prostate screening exam.

19. The method of claim 17, wherein the prostate screening exam is a digital rectal exam and/or PSA test.

20. The method of claim 10 or 19, wherein the PSA level is between the range of 2.5-10 ng/mL.

21. The method of claim 17, wherein the prostate screening exam further comprises one or more of the steps of taking one or more of prostate biopsies, prostate ultrasound, MRI fusion, imaging, Prostate Health Index (PHI), kallikrein test, blood test, prostate cancer antigen 3 (PCA3) test, and/or epigenetic test.

22. The method of claim 15, wherein the step of obtaining a sample further comprises determining the grade and/or aggressiveness of the cancer.

23. The method of claim 22, wherein determining the grade of cancer further comprises assigning a Gleason score, Gleason sum, Grade Group, and/or genomic testing.

24. The method of claim 23, wherein the Gleason score is 1, 2, 3, 4, 5, 6, 7, 8, 9, or 10.

25. The method of claim 24, wherein the Gleason score indicates the group of well-differentiated or low-grade, moderately-differentiated or intermediate-grade, or poorly-differentiated or high-grade cancer.

26. The method of claim 23, wherein the Grade Group is 1, 2, 3, 4, or 5.

27. The method of claim 15, wherein the step of obtaining a sample further comprises determining the stage and/or spread of the cancer.

28. The method of claim 27, wherein the spread of the cancer includes one or more of metastasis to the bones, lungs, lymph nodes, liver, brain, adrenal glands, breasts, eyes, kidneys, muscles, pancreas, salivary glands, spleen, stomach, colon, testicles, anus, skin, esophagus, intestine, and/or blood.

29. The method of claim 27, wherein the step of determining the stage and/or spread of cancer further comprises one or more of a bone scan, ultrasound, computerized tomography (CT) scan, magnetic resonance imaging (MRI), positron emission tomography (PET) scan, prostate-specific membrane antigen (PSMA) test, advanced ultra-high-field scanning technology.

30. The method of any one of claims 1-8, wherein the individual in need of treatment is at increased risk for prostate cancer.

31. The method of claim 30, wherein the increased risk for prostate cancer is associated to one or more of a family history of prostate cancer, family cancer syndrome, family history of breast, ovarian, colon, and/or prostate cancer, age, chemical exposure, Agent Orange exposure, obesity, regular consumption of high-fat foods and/or processed carbohydrates, sedentary lifestyle, genetic factors, enlarged prostate, and/or prostatic intraepithelial neoplasia.

32. The method of claim 31, wherein the genetic factors comprise one or more of RNASEL, BRCA1, BRCA2, MSH2, MLH1, HOXB13, HPXC, CAPB, ATM, FANCA, HPC1, and HPC2.

33. The method of any one of claims 1-32, wherein the change in biomarker level is detected by one or more of the methods of mass spectrometry, chromatography, high-performance liquid chromatography, spectroscopy, ELISA, or immunoassay.

34. The method of any one of claims 1-32, wherein the method further comprises the step of predicting the cancer status of an individual.

35. The method of claim 34, wherein the step of predicting cancer status further comprises the use of one or more of the prostate specific antigen score, Gleason score, and/or body mass index.

36. The method of claim 34, wherein the prediction of cancer status is made by a machine learning classifier.

37. The method of claim 36, wherein the machine learning classifier has been trained using biomarkers levels obtained from individuals with prostate cancer and control individuals that do not have cancer.

38. The method of claim 36, wherein the machine learning classifier is a random forest.

39. The method of any one of claims 1-3, wherein the step of treating the individual comprises one or more of active surveillance, surgery, cancer treatment, and/or one or more cancer treatments.

40. The method of claim 39, wherein the cancer treatment comprises one or more of surgery, radiation, proton therapy, hormone therapy, chemotherapy, immunotherapy, bisphosphate therapy, cryotherapy, ultrasound, and/or palliative care.

41. The method of any one of claims 1-3, wherein the cancer treatment comprises continuing the therapy, ceasing therapy, or changing the method of therapy.

42. A method of determining the grade and/or aggressiveness of a cancer, comprising identifying a change in the level of biomarkers that consist essentially of Methionine, Homocysteine, Glutamic acid, Ornithine, Inosine, and/or a combination thereof.

43. A method of determining the grade and/or aggressiveness of a cancer, comprising identifying a change in the level of biomarkers that consist essentially of Methionine, Homocysteine, Glutamic acid, Ornithine, Inosine, N-Acetyl Aspartate, Glutamine, Sarcosine, Succinate, Malate, and/or a combination thereof.

44. A method of determining the grade and/or aggressiveness of a cancer, comprising identifying a change in the level of biomarkers that consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate, Fumarate, and/or a combination thereof.

45. The method of any one of claims 1-44, wherein the method further comprises determining the grade and/or aggressiveness of the cancer by analyzing a sample from an individual.

46. A method of determining the stage and/or spread of the cancer comprising identifying a change in the level of biomarkers that consist essentially of Methionine, Homocysteine, Glutamic acid, Ornithine, Inosine, and/or a combination thereof.

47. A method of determining the stage and/or spread of the cancer comprising identifying a change in the level of biomarkers that consist essentially of Methionine, Homocysteine, Glutamic acid, Ornithine, Inosine, N-Acetyl Aspartate, Glutamine, Sarcosine, Succinate, Malate, and/or a combination thereof.

48. A method of determining the stage and/or spread of the cancer comprising identifying a change in the level of biomarkers that consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate, Fumarate, and/or a combination thereof.

49. The method of any one of claims 1-48, wherein the method further comprises determining the stage and/or spread of the cancer by analyzing a sample from an individual.

50. A method of predicting the cancer status of an individual comprising identifying a change in the level of biomarkers that consist essentially of Methionine, Homocysteine, Glutamic acid, Ornithine, Inosine, and/or a combination thereof.

51. A method of predicting the cancer status of an individual comprising identifying a change in the level of biomarkers that consist essentially of Methionine, Homocysteine, Glutamic acid, Ornithine, Inosine, N-Acetyl Aspartate, Glutamine, Sarcosine, Succinate, Malate, and/or a combination thereof.

52. A method of predicting the cancer status of an individual comprising identifying a change in the level of biomarkers that consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate, Fumarate, and/or a combination thereof.

53. The method of any one of claims 1-52, wherein the method further comprises determining the stage and/or spread of the cancer by analyzing a sample from an individual.

54. The method of any one of the preceding claims, wherein the cancer is indolent or aggressive.

55. A method of treating an individual in need thereof comprising the steps of detecting a change in the level of a combination of biomarkers and treating the individual in need thereof with at least one cancer therapy, wherein the biomarkers comprise, consist of, or consist essentially of Methionine, Homocysteine, Glutamic acid, Ornithine, Inosine, and/or a combination thereof.

56. A method of treating an individual in need thereof comprising the steps of detecting a change in the level of a combination of biomarkers and treating the individual in need thereof with at least one cancer therapy, wherein the biomarkers comprise, consist of, or consist essentially of Methionine, Homocysteine, Glutamic acid, Ornithine, Inosine, N-Acetyl Aspartate, Glutamine, Sarcosine, Succinate, Malate, and/or a combination thereof.

57. A method of treating an individual in need thereof comprising the steps of detecting a change in the level of a combination of biomarkers and treating the individual in need thereof with at least one cancer therapy, wherein the biomarkers comprise, consist of, or consist essentially of Melatonin, gamma-Aminobutyric acid, Isoleucine, Adenosine, Putrescine, Arginine, Ornithine, Homocysteine, Valine, Methionine, Kynurenine, Inosine, Proline, Glutamic acid, Sarcosine, Glutamine, Kynurenic acid, reduced Glutathione, Pyruvate, Lactate, alpha-Ketoglutarate, succinate, N-Acetyl Aspartate, 2-Hydroxy Glutarate, Malate, Fumarate, and/or a combination thereof.

58. A method of identifying a subject for treatment of prostate cancer comprising the step of identifying a change in the level of a combination of biomarkers comprising, consisting of, or consisting essentially of the metabolites Methionine, Homocysteine, Glutamic acid, Ornithine, Inosine, and/or a combination thereof from the sample of the individual, predicting cancer status of the individual, and determining treatment for the individual.

59. A kit comprising the composition of any one of the claims 1-58, said composition housed in a suitable container.
